(19) United States
(12) Patent Application Publication
(10) Pub. No.: US 2026/0096843 A1
(43) Pub. Date: Apr. 9, 2026

(54) GENERATOR CONTROL FOR A SURGICAL INSTRUMENT

(71) Applicant: Erbe Elektromedizin GmbH, Tuebingen (DE)

(72) Inventors: Tjark BRANDT, Kusterdingen (DE); Sandra KELLER, Bisingen (DE); Sebastian NAGEL, Herrenberg (DE)

(21) Appl. No.: 19/351,181

(22) Filed: Oct. 6, 2025

(30) Foreign Application Priority Data
Oct. 8, 2024 (EP) 24205269.4
Sep. 9, 2025 (EP) 25201124.2

(51) Int. Cl. A61B 18/12; A61B 18/00

(52) U.S. Cl. CPC A61B 18/1233; A61B 2018/00482; A61B 2018/00577; A61B 2018/00666; A61B 2018/00708; A61B 2018/00738; A61B 2018/1213

(57) ABSTRACT

With the generator according to the invention the treatment of biological tissue by means of electrosurgical instruments, particularly by means of argon plasma probes, can be carried out reliably without depending on the personal skills of a treating person. With the aid of image-supported measurement value generation, for example using a camera or a medical imaging device, such as CT, a multiplicity of test treatments of tissue samples is carried out and on this basis a training data set is created. From the training data set based on machine learning a control data set is created that controls during subsequent use an apparatus located in an operation room without the aid of a camera observation of the field of operation. Only the typical pattern of sensor data are evaluated that have been assigned to specific tissue effects during the camera-monitored training sessions.

This application claims priority to European Patent Application No. 24205269.4, filed Oct. 8, 2024, and European Patent Application No. 25201124.2, filed Sep. 9, 2025, the entirety of each of which are incorporated herein.

The invention refers to an artificial intelligence (“AI”) generator for operating a surgical instrument, wherein the generator comprises a control module based on artificial intelligence or comprising artificial intelligence.

Electrosurgical generators serve to supply electrosurgical instruments with current and, if necessary, additional operating media, such as fluids, for example argon, CO₂, NaCl solution etc. Thereby the control of the electrical generator is of particular importance, because by means of the control the energy introduction in the biological tissue and thus the occurring surgical effect can be directly controlled. However, the relations between the variables characterizing the electrical current, such as frequency, voltage, current strength, power, modulation type, crest factor as well as the electrical variables of the load formed by the biological tissue, such as impedance, and the surgical effect that can be achieved are complex. For this reason, in the past different approaches have been tried to use concepts of artificial intelligence for generator control. In this regard US 2023/0071343 A1 describes a computer-implemented method that uses electrical variables, switching conditions of an instrument and image data obtained from a camera for training purposes. Also, during later use of a respective trained module this module uses currently detected electrical variables and in addition image data in order to estimate whether the current settings result in a successful application.

From US 2020/0265309 A1, an algorithm for estimation of tissue parameters is known that is based on machine learning. Based on electrical parameters a tissue parameter is determined used to control the energy output.

DE 10 2020 105 835 A1 discloses an apparatus for carrying out endoscopic surgeries, wherein an optical recording device is part of this apparatus the field of view of which is directed onto the treated tissue or the tissue to be treated by means of the RF electrode. Prior to or during the treatment of the tissue a classification of the tissue type of the tissue in the area of the RF electrode is carried out based on optical measurement signals of the recording device and based on the result of the optical classification an RF mode is set that matches with the determined tissue type.

Further prior art is formed by DE 10 2021 101 410 A1, US 2023/0420032 A1, EP 4 134 029 A1 and EP 3 541 313 A1.

For the electrosurgical treatment of tissue, particularly argon plasma coagulation, the treating person needs considerable experience in order to be able to already assess during the execution of the surgery the result that is later visible, that means the effect achieved on the tissue. The plasma acting on the tissue emits light itself and thus can create blinding effects. In addition, the emitted light can complicate a purely visual evaluation of the obtained result due to its spectral composition. Also fume production, vapor production or also a partial coverage of the treatment site by the instrument or tissue parts can complicate the evaluation of the result during treatment.

Starting therefrom it is an object of the invention to provide a generator with which an instrument can be operated, so that a desired effect can be achieved on the tissue, for example a desired penetration depth of the effect in the tissue, independent from the visual observation by the treating person and largely independent from the personal ability of the treating person.

This object is solved, in one example, by means of a generator according to claim 1:

The generator according to the invention comprises a control module connected with sensors for electrical variables. The electrical variables are predominantly and preferably exclusively derived from the current and the voltage with which the electrical instrument is supplied that is connected to the generator. The electrical variables can be supplied by sensors, for example current sensors, voltage sensors, sensors for determination of a power factor, sensors for determination of a non-linearity of the connected load and so on. The control module for the operation of the generator receives however exclusively electrical variables, but not image data, that can come from an imaging device, such as a camera or a CT device. Rather the control module can be “blind” during evaluation of the effect of the surgical application. The control module thereby operates based on an AI module that creates a control data set in a training run. During the training, the electrical variables already mentioned above enter into the training data that are also monitored later during application on the patient.

During the training, particularly a set of image data can be used in addition to the electrical variables that contains information about the penetration depth of a tissue effect resulting from the treatment, such as a coagulation or ablation. For example, this penetration depth can be provided in numerical form as effect label, as profile or in color-coded manner and can be linked with the detected electrical variables as additional training information. The expression “image data” in this application include measurement data of cameras as well as image-like illustrations that are gained from measurement values of non-imaging methods. For example, point-shaped or two-dimensionally shaped spectral measurement data from a diffuse reflection spectroscopy device can be processed into parametric two-dimensional maps, which form image data in the sense of this description.

The sensors considered in the training data set do not have to be identical with the sensors connected to the control module during operation. Preferably, the measurement values for the training data set are provided by other sensors of similar type that detect electrical values of the same type. It is thereby possible to create the training data set independent from the specific configuration of the sensor technology used during operation.

The image data can be individual pictures that are recorded upon achieving a desired effect level and can be stored. Alternatively, image data can comprise multiple images gained with temporal distances or also video sequences. For the training, image data from an external camera can be used that records the treatment and the treatment progress. Alternatively or additionally, image data may not come from a camera, but from a medical imaging device, for example a computer tomography device (CT device), a magnetic resonance tomography device (MRT device), an ultrasonic device, an optical coherence tomography device (OCT device) or a diffuse reflection spectroscopy device (DRS device). The image data used in this embodiment can contain in addition information about the penetration depth.

Moreover, tissue effects obtained on the biological tissue during training are classified and assigned with a respective effect label. In the sense of this application effect label means each target variable that is relevant for treatment success including qualitative parameters such as coagulation or ablation degree as well as quantitative parameters such as the achieved penetration depth of the tissue effect. This can be carried out manually or automatically.

In a further particular embodiment image data serve as source of information about the penetration depth of a tissue effect created by the treatment, such as a coagulation or ablation. The depth effect can be provided in form of a numerical value, a depth profile or a color-coded illustration within a two-or three-dimensional image. The measurement of the penetration depth can be based on changes of tissue characteristics that are related to the coagulation or ablation effect. Such changes can be of optical, acoustical, electrical or chemical nature and can be detected by means of known medical measurement methods. The penetration depth determined in this manner is considered when assigning a label and is part of a respective effect label that is linked with the detected electrical variables.

The tissue influence can be carried out by means of argon plasma coagulation during which energy is transmitted via an ionized argon gas without direct electrode contact. Alternatively, or additionally, a contact coagulation can be used during which the electrode is in direct contact with the tissue surface during energy output. Both methods are suitable for producing a penetration depth of the tissue effect, which can be detected as part of an effect label, and can be combined with the control according to the invention.

The AI module is configured to determine the control data set from the training data set by means of machine learning, wherein the control data set indicates which electrical variables or which pattern of electrical variables has to be present in order to achieve a tissue effect corresponding to a desired effect label, that means, for example, a desired penetration depth of the tissue effect. In a first embodiment the pattern of electrical variables thereby comprises predefined values or value ranges for each monitored electrical variable. In an extended embodiment the pattern of electrical variables comprises individual values of the electrical variables obtained in time intervals or temporal progresses of each monitored electrical variable. In another more elaborate embodiment the pattern comprises the monitored electrical variables at least at two different power output conditions of the generator. The different power outputs can be realized by different voltages, currents, current limitations, forms of modulation or the like. Different forms of modulation can be, for example, continuous wave (CW), amplitude modulation, pulse-pause sampling with constant or variable pulse-pause ratio. These different power outputs can define different modes that create different surgical effects. In addition, the pattern can comprise the temporal progresses of one or more electrical variables prior to and after switching the generator between different power outputs or modes. With these different approaches different requirements on the accuracy of the treatment result can be complied.

The two modes mentioned above distinguish preferably at least with regard to the electrical power output to the instrument. While in the first mode a high power can be output, the second mode is characterized by lower power. Preferably the power in the first mode is dimensioned so that it results in a devitalization and coagulation of biological tissue and related thereto to a quick attainment of the desired tissue effect, preferably having a predefined penetration depth. The power is preferably higher than 10 W. The power in the second mode is preferably dimensioned so that it does not produce a surgical effect on the tissue, particularly no devitalizing effect. The power can be limited to values of 10 W or less.

Preferably, the control module having the IA module is configured to switch from the first mode into the second mode upon determination of a pattern of electrical variables according to any of the embodiments above, to which pattern a desired effect label is assigned. Optionally, it can be configured to thereby further monitor the pattern of the electrical variables also after switching into the second mode. This particularly allows to take account of the non-linear electrical characteristics and the time-dependent electrical characteristics of biological tissue. For example, amongst others, the tissue impedance may even change after the influence with high power also during the second mode with low power. For example it may decrease due to re-wetting of previously dried-out tissue sections.

In addition, the control module can be configured to switch back from the second (weak) mode into the first (strong) mode if a pattern of electrical variables detected in the second mode corresponds to an effect label being too low, for example a too low penetration depth of the effect in the tissue of the patient and/or a too low tanning degree. Vice versa it can also be configured to switch from the second (weak) mode into a third mode if the pattern detected in the second mode corresponds to an effect label being too high, for example a too deep penetration depth of the effect in the tissue of the patient and/or a too high tanning degree. Per se this situation is an unexpected and normally not intended case, because tissue changes due to electrical influence are largely irreversible. The third mode can be a switch-off mode impeding a still further access of the intrinsically desired tissue effect. The tissue effect is a change of the tissue, for example a coagulation.

The control module can comprise a distance measurement function. It can serve to block generation of data, that is electrical variables of sensors, that are difficult to interpret or can no longer be interpreted. If the distance between the instrument and the tissue is too long, signal distortions can occur due to the (too) high influence of the electrical non-linearity of the arc or plasma provided between the instrument (particularly its electrode) and the tissue, which affect or prevent a reliable signal interpretation. For example, the distance determination can be carried out by way of a pattern recognition in the context of which specific patterns of electrical variables which are based on too long discharge distances (plasma) are recognized, for example for requesting the surgeon to reposition the instrument.

The invention is also directed to the method for training a control module and its AI module of an electrical generator and to the subsequent operation of such a generator as mentioned above. This method comprises a training progress in the context of which a training data set is generated. The latter is preferably obtained with a non-varying setting, which is predefined for the generator. With this setting the instrument supplied by the generator influences the tissue, whereby the electrical variables resulting therefrom, particularly their temporal progresses and values as well as resulting tissue changes are detected that can be recorded by means of a camera in the form of pictures, picture sequences or video sequences. Additionally or alternatively, also the depth of the tissue change can be detected in the form of measurement data, which can comprise the image sequences, video sequences, spectral progresses and the like, for example by means of a CT, MRT, ultrasonic, OCT or DRS imaging device. The treated tissue samples are inspected automatically or manually and are classified in terms of the obtained tissue effect and/or in terms of the penetration depth of the attained tissue effect. In accordance therewith effect labels are assigned to the images, image sequences and/or video sequences and the gained pattern of the electrical variables. From the entirety of the patterns of the gained electrical variables the images, image sequences and/or video sequences and the assigned effect labels (training data set) a control data set is generated that only represents a relation between the electrical variables and the effect labels. Thus, by means of the control data set without the aid of a camera image or another image produced by imaging, the electrical influence on the biological tissue is controlled so that the treatment result corresponds to the desired effect label.

In doing so, a generator is provided comprising a possible selection for the desired effect strength in a specific mode just as conventional generators. However, the effect strength is now no longer a value on an abstract scale, for example from 1 to 10, assigned to an output power, but a preset of treatment results. For example, the effect strengths can be preset multiple levels, for example four levels (low coagulation, weak coagulation, strong coagulation, very strong coagulation). If the operator selects weak coagulation, the generator operates in the first mode based on the control data set until the weak coagulation is attained and then switches to the second mode in which no further coagulation is achieved. If the operator selects strong coagulation, the generator first operates in the first mode as well, indeed until the strong coagulation is attained, whereupon it then switches in the second mode. In doing so, the treatment result is largely independent from the personal skill of the operator. The operator can always attain the desired surgical effect on the tissue located in front of him/her and indeed independent from the subjective optical evaluation of the change procedures on the tissue during treatment.

In a specific embodiment the invention comprises a method for coagulation and/or ablation of tissue of a patient, particularly mucosa tissue. The method particularly serves for large area surface treatment, as it is advantageous, for example, for the therapy of obesity, Barrett's esophagus, endometriosis or similar diseases, in which tissue effect over an area as large as possible is desired. For example, in doing so an ablation and/or coagulation of the gastric mucosa can be carried out. For example, the method comprises the following steps:
inserting an endoscopically guided electrosurgical instrument to a tissue region of the patient's body to be treated, for example the stomach of the patient;
selecting a desired tissue effect, comprising at least a desired penetration depth of the tissue effect and/or a desired degree of devitalization of the tissue for the tissue section to be treated;
detecting electrical variables during the treatment by the instrument and determining the current tissue effect, particularly the current penetration depth and/or the current degree of devitalization of the tissue based on these electrical variables and a control data set that has been generated in advance by means of machine learning, wherein the control data set has been gained from a training data set which comprises electrical variables and the target variables assigned thereto;
controlling the power output of the instrument depending on a specific tissue effect so that the desired tissue effect is attained and subsequently the power output of the instrument is automatically reduced or terminated in order to avoid an excess.

The indicated method steps can also be carried out in another sequence or at least partially parallel, as far as it is technically useful and expedient.

With the above method particularly damages in the depth of the treated tissue can be reduced down to a very low amount, so that, for example, only the superficial tissue layer is treated, for example the superficial mucosa layer. Depending on the treatment, the superficial tissue layer can contain Ghrelin-cells that are of importance for obesity treatment, or can contain focuses of endometriosis.

The desired penetration depth of the tissue effect can be set prior to the ablation and/or coagulation of the tissue. Particularly, the method does not require additional surgical treatments such as the introduction of a material between the mucosa and the submucosa or in the submucosa for protecting the submucosa.

In preferred embodiments the penetration depth corresponds approximately to two-thirds, preferably half or one-fourth of the submucosa thickness, for example.

Additionally or alternatively, scarring in the stomach wall of the patient can be created that reduces the elasticity of the stomach wall and thereby the storage capacity of the stomach, whereby a desired degree of scarring can be set and attained. The use of a control data set trained on mucosa tissue allows a specifically precise setting of the energy output for the specific characteristics of the stomach mucosa.

Additional details and advantageous embodiments of the invention are derived from the following description and the drawing. The drawing shows:

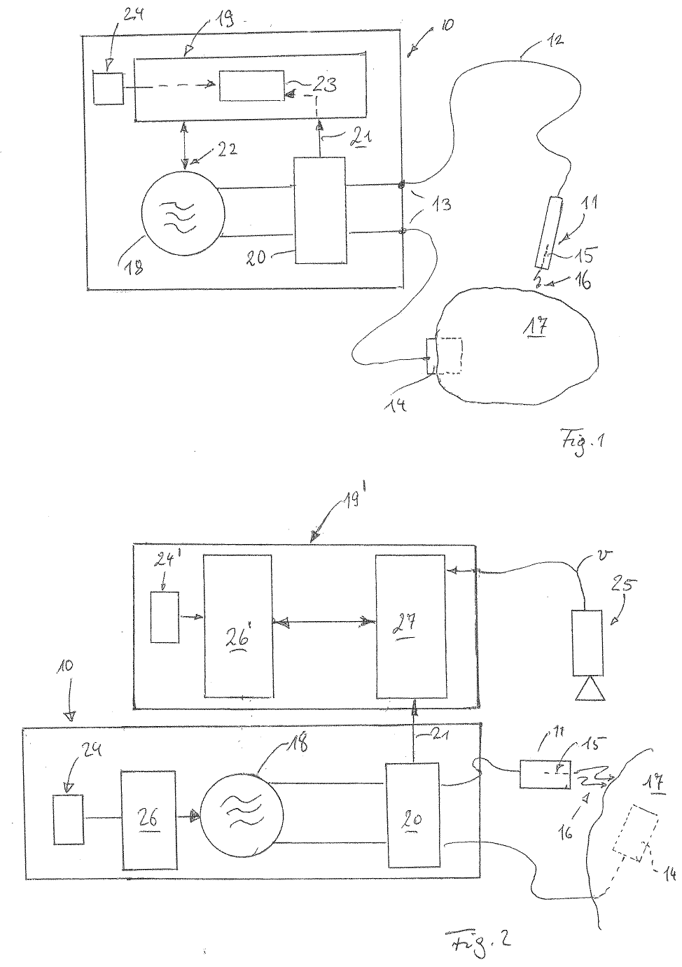

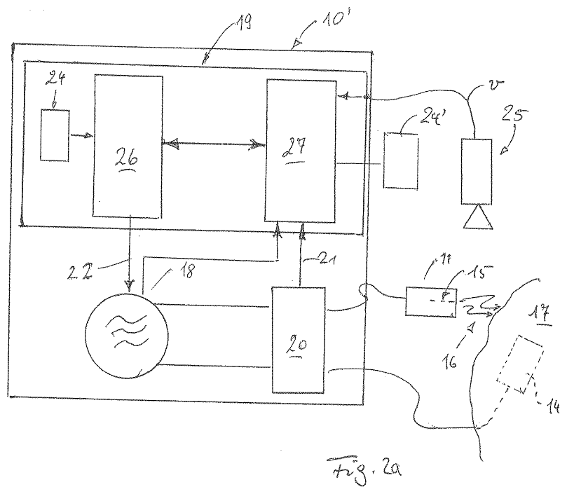

FIG. 1 the generator according to the invention, the instrument connected thereto and a biological object during a surgical procedure in a schematic illustration,

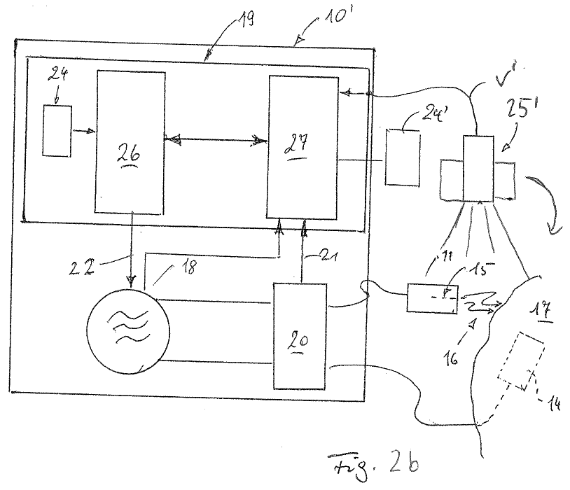

FIG. 2 an example of a generator for producing a training data set with a camera and an instrument connected thereto during influence on a biological object in schematic illustration,

FIG. 2a another example of a generator for producing a training data set with a camera and an instrument connected thereto during influence on a biological object in schematic illustration,

FIG. 2b another example of a generator for producing a training data set with a CT device and an instrument connected thereto during influence on a biological object in schematic illustration,

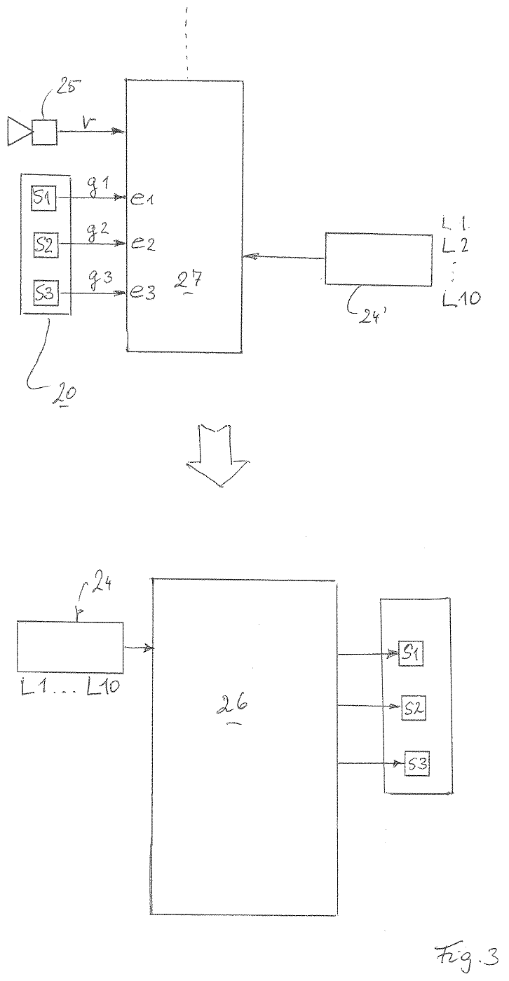

FIG. 3 block diagrams for illustration of gaining a training data set and therefrom,

FIG. 3a block diagrams for illustration of gaining a training data set and therefrom using a CT device instead of a camera,

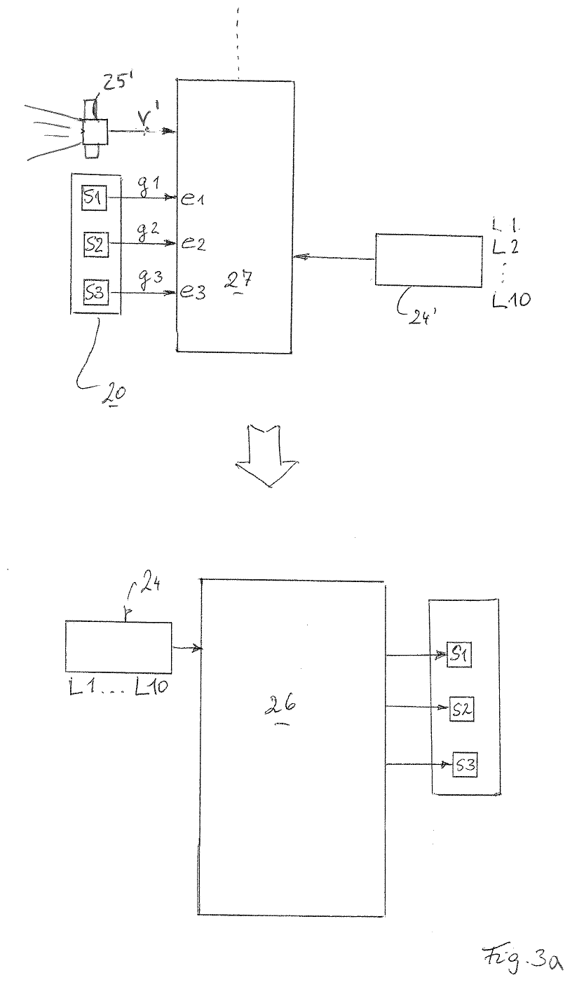

FIG. 4 different patterns of gained electrical variables during treatment of biological tissue in form of a diagram,

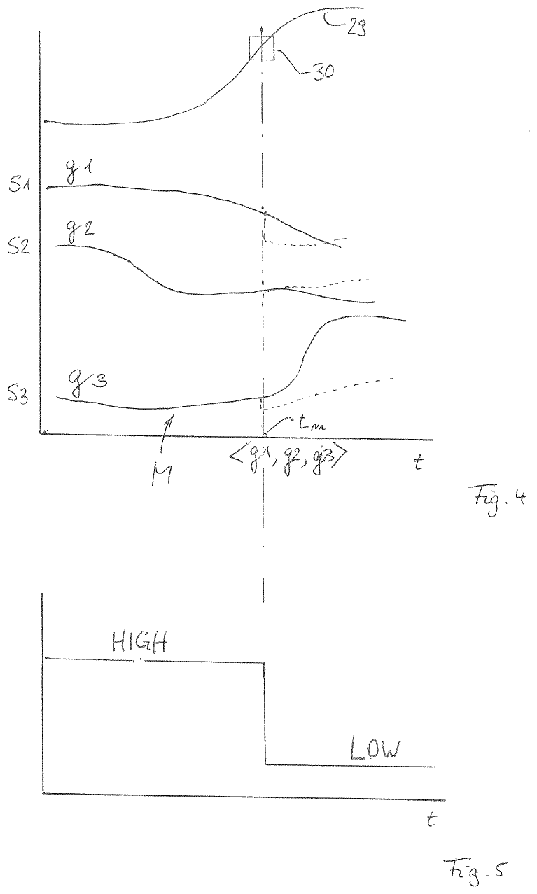

FIG. 5 a diagram for illustration of switching the generator between different modes.

In FIG. 1 a generator 10 according to the invention is illustrated to which an instrument 11 is connected, particularly an argon plasma instrument. Via a cable 12, this instrument 11 is connected with a generator output 13 to which also a neutral electrode 14 is connected for guiding back a current output to the instrument 11. This structure applies for monopolar instruments. In case of bipolar instruments both poles of the output of generator output 13 are connected to the instrument 11. According to an example a control module 19 can control the degree of the effect that can be achieved on a tissue, that means, for example, the tanning or devitalization degree of the treated tissue and/or a preset penetration depth of the tissue effect. Thereby the control is based on a control data set that has been created using training data. For example, the training data set can be produced on a defined tissue type, for example mucosa tissue.

If the instrument 11 is specifically an argon plasma instrument, it is additionally supplied from the generator 10 or another supplying apparatus with gas, particularly argon, which is however not illustrated in FIG. 1 for sake of clarity. Thereby in the training data set and thus in the control data set also parameters of the gas flow can be included.

The instrument 11 comprises an electrode 15 that can be arranged in a gas conveying channel, particularly argon conveying channel and is illustrated in FIG. 1 in dashed lines. From this electrode 15 and supplied by generator 10 a plasma discharge 16 originates inside which electrical current flows from the electrode 15 to the biological tissue 17, to which the neutral electrode 14 is connected. This electrical current applies a thermal effect on the biological tissue.

For supply of instrument 11, that means for providing electrical power at the output 13, generator 10 comprises a source 18, particularly an RF source for high frequency electrical voltage that can be controlled by means of a control module 19. Particularly control module 19 can thereby control selected electrical variables, for example the modulation, the amount (amplitude) of the voltage, the current strength and similar.

For example, source 18 is formed by an oscillator oscillating with high frequency and configured to output a voltage of multiple 1000 Vₚₑₐₖ (peak-to-peak voltage) and an electrical power of multiple Watts, preferably >10 W, for example 100 W, at the output 13. For detection of electrical characteristic variables, such as the voltage or the current, the phase angle between the voltage and the current, the non-harmonic distortion of the current, etc. serves a sensor block 20 that provides the measured variables to the control module 19 as indicated by an arrow 21. The arrow 21 marks the direction of information flow and therefore comprises only one arrow tip end. However, it is also possible to configure the embodiment so that control module 19 specifically requests sensor data and transmits data queries for this purpose to sensor block 20.

The control module 19 controls the oscillator 18, which is symbolized by an arrow 22. In addition, the control module 19 can receive information from the oscillator 18 that does not depend on the surgical effect attained on the tissue 17. For example, such information can be information about the oscillating frequency of oscillator 18 or its modulation type (for example continuous wave (CW) or pulsed, for example on/off-sampled).

The control module 19 comprises a control data set, which has been created by machine learning from a training data set. The control data set is configured to establish a relation between effect strengths or effect levels, including a desired penetration depth of the tissue effect, and electrical variables, which are provided by oscillator 18 and/or sensor block 20. The respective effect strengths or tissue effects including the penetration depth are preset using an input device 24, which is part of generator 10 or which is configured separate therefrom, for example by a mobile device, such as a tablet, mobile phone or the like.

Particularly the input device 24 can also be part of the instrument 11.

As apparent, the control of generator 10 is solely based on the effect strength and/or a desired penetration depth of the tissue effect preset by means of the input device 24 as well as on electrical variables from the oscillator 18 and/or sensor block 20. A camera for inspection of an effect attained on the biological tissue 17 is neither provided nor necessary. Different to conventional apparatuses the effect strength or the desired penetration depth preset by means of the input device characterizes the effect to be actually attained on the biological tissue, which is characterized, for example, by a tanning degree of the tissue or a specific penetration depth into the tissue. As soon as the desired effect is attained, the generator switches into the second mode in which no further tissue influence is carried out. This is done without requiring a camera image of the treated tissue. For this reason, the desired effect is attained, but not exceeded, even if the plasma jet is directed onto a tissue area for an unnecessary long time.

FIG. 2 illustrates the generation of a training data set 27 on the basis of which subsequently the control data set is created by AI module 23. For this purpose a separate training module 19′ is provided. Image data v from a camera 25 are supplied to the training module 19′ in addition to the variables generated by sensor block 20, wherein the camera field of view comprises particularly the location of the tissue 17, which is influenced by discharge 16. The training module 19′ serves for generation of a training data set 27. In addition, in the training module 19′ a training input means 24′ is provided via which a person in charge of generation of the training data set 27 can input the attained effect. For example, if multiple tanning stages, for example 10 tanning stages, of the tissue are distinguished they can have a range starting with no tanning up to a beginning carbonization. During training this tanning degree is assigned to the patterns of the electrical variables, which have been generated using sensor block 20 or have been directly received from oscillator 18, as effect label in the training data set 27. In addition, different tissue types may be set using training input means 24′. The training module 19′ additionally comprises a block 26′ configured to create and/or store control signals for an oscillator 18 and a power output of generator 10 to the instrument 11 associated therewith. The control signal can be assigned to different modes, which are used during generation of training data set 27. In a first mode oscillator 18 outputs a high power to the instrument 11, producing a surgical effect on the tissue 17, for example a visible coagulation. In a second, weaker mode the power output from the oscillator 18 to the instrument 11 is reduced to such an extent that the discharge 16 does no longer attain a visible effect on the surface of tissue 17.

In FIG. 2, generator 10 comprises an input device 24 and a block 26. Via input device 24 a person in charge of generating the training data set 27 can input the effect attained by means of the discharge 16 influencing the tissue 17 for test purposes. The block 26 is configured to control the oscillator 18 and thus the power output of generator 10 to the instrument 11.

FIG. 2a illustrates another example of generator 10′ for producing a training data set. For the example shown in FIG. 2a, the explanations provided in relation to FIG. 2 with reference to the reference signs apply accordingly. The example shown in FIG. 2a distinguishes from the example shown in FIG. 2 substantially in that training module 19′ is not provided separate from generator 10′.

FIG. 2b illustrates another example in which a computer tomography device 25′ is used as imaging device instead of a camera 25. The explanations in relation to FIG. 2 and FIG. 2a with reference to the reference signs apply accordingly for the example shown in FIG. 2b. The CT device 25′ determines during or after tissue influence image data v′ as measurement data from which, inter alia, the penetration depth is derived. The penetration depth can comprise continuous values or different discrete penetration levels, for example ten levels. The determined depth values are linked in form of effect labels with the electrical variables detected by means of sensors S1 to S3 and are taken over in the training data set 27. In the illustrated example, training module 19′ is integrated in the generator 10 as in FIG. 2a, however, it can also be configured as separate unit as in FIG. 2. On the basis of these training data control module 19 can be controlled subsequently so that the predefined effect depth d is exactly attained, however not exceeded.

FIG. 3 illustrates the generation of the training data set into which video sequences or individual pictures of camera 25 as well as data from the sensor block 20 is comprised. These data can be the outputs g1, g2, g3 of one or multiple sensors, for example the sensors S1, S2, S3, which, for example, characterize the amount of the current, the amount of the phase angle between current and voltage, the crest factor and/or the non-linearity of the current. Additional variables can be determined as well. The aforementioned variables are only examples. In a first embodiment the variables detected by sensors S1 to S3 can be detected only at the end of the influence. The variables thus form a static pattern without temporal parameter. However, it is also possible to detect temporal progresses. This applies for the camera 25 as well as the sensor block 20 so that the temporal progresses of the respective variables can be detected. Top of FIG. 3 thereby illustrates that data from the sensor block 20 as well as the camera 25 as well as the input means 24′ are combined for generation of the training data set 27. While data from camera 25 and sensor block 20 characterize the current condition of the optical and electrical variables, the effect label L1 . . . L10 input via the input means 24′ characterizes the attained result.

For generation of the training data set 27 a multitude of sample treatment processes is carried out during which a suitable specimen is electrically influenced by means of the device according to FIG. 2. Using machine learning the control data set 26 illustrated on the bottom of FIG. 3 is determined therefrom. The latter comprises the desired effect as input variable via the input device 24 (for example medium tanning). From the information of the training data set it readily has the respective values that the sensors S1 to S3 need to have for attaining the desired effect. The training data set 27 can comprise effect labels that characterize visible tissue changes as well as quantitative parameters, such as the penetration depth of the attained tissue effect. The control data set 26 monitors now sensors S1 to S4 with regard to attaining the predefined values. Thereby control module 19 operates as follows:

The oscillator 18 can at least be operated in a first mode with high power attaining a surgical effect on the tissue 17 as well as with a second, lower power that does not attain a surgical effect. During generation of the training data set with the device according to FIG. 2, oscillator 18 is first operated in the first mode and then switched off or transitioned into the second mode, whereafter the effect label is defined according to the attained treatment result. The effect label can comprise qualitative features, such as the visible degree of the tissue change (for example tanning) and/or quantitative features, such as the attained penetration depth of the tissue effect. During the actual treatment of a patient using the device according to FIG. 1, control module 19 now operates using the control data set in the first mode at the beginning of the treatment. The actually attained, however for the surgeon not well recognizable transformation (for example tanning) of tissue 17 is illustrated by a top curve 29 in FIG. 4. As apparent the tanning increases over time until a maximum is reached. The respective variables of sensors S1, S2, S3 as well as additional sensors can have different temporal progresses. For example, the value g1 of sensor S1 can represent the current that can have a decreasing progress. The value g2 of sensor S2 can be a humidity value, for example, wherein the tissue humidity can decrease over time and with treatment progress. A third value g3 of sensor S3 can be any other electrical variable or a variable characterizing the tissue 17 calculated from the electrical variables.

FIG. 3a shows another training scenario in which the image recording is carried out using a CT device 25′. The CT device 25′provides image data from which the penetration depth d of the attained tissue effect is determined. The depth indication as a component of the effect label is assigned to the electrical variables g1 to g3 concurrently detected by sensors S1 to S3 and entered into the training data set 27. This embodiment can be combined with a training module 19′ integrated in generator 10 as well as a training module embodiment separate from generator 10.

In the embodiment of FIG. 3 the treating person has preset an effect, that means a tanning degree, which is illustrated in FIG. 4 as tolerance field in a box 30. In the embodiment of FIG. 3a the treating person has preset a respective effect depth, that means the (maximum) penetration depth of the tissue effect. In the control data set 26 the value characterized by box 30 is assigned to the characteristic progresses and thus patterns of the electrical variables monitored by sensors S1, S2, S3, which are illustrated in FIG. 4. The pattern can be the combination of the variables g1, g2, g3 of sensors S1, S2, S3 at one point in time tm (measurement point in time). However, the pattern can also comprise sections of the temporal progresses or the entire temporal progresses of the variables of the three sensors S1, S2, S3. As soon as this pattern is recognized control module 19 reduces the energy output to the instrument 11 from a high value HIGH to a low value LOW illustrated in FIG. 5.

With generator 10 according to the invention the treatment of biological tissue by means of electrosurgical instruments, particularly by means of argon plasma probes, can be carried out in a reliable manner without having to rely on the personal skills of a treating person. Using a camera-supported measurement value generation a multiplicity of test treatments of tissue samples is carried out and based thereon a training data set is created. From the training data set a control data set is generated using machine learning, wherein the control data set controls an apparatus 10 in an operation room in the subsequent use without the aid of camera observation of the operation field. Only the typical pattern of sensor data are evaluated that are assigned during camera-monitored training sessions to specific tissue effects. The attainment of the preset value can thereby be determined based on the effect degree and/or the penetration depth d.

By means of the invention it is in addition possible to produce the thermal influence on the tissue very quickly (as quick as possible) and in controlled manner (no overdosage). For this control module 19 operates in the application phase with two operating conditions (modes), namely a condition with high power and a condition with low power. The high power condition (first mode) is used in order to achieve the desired thermal effect on the tissue. The low power condition (second mode) is however not intended for achieving a thermal effect. When the plasma—that serves for contactless transmission of power onto the tissue—is ignited in the first mode and a current flows, electrical data are detected (here: peak voltage Up, peak current Iₚ, effective (root mean square) voltage Uᵣₘₛ, effective (root mean square) current Iᵣₘₛ, power factor, frequency, resistance, spark creation) that serve as input data for the control module 19. On the basis of the prediction of control module 19 the oscillator is automatically switched between first mode and second mode in order to immediately limit the tissue damage and to attain the desired degree of devitalization. If the tissue damage predicted by control module 19 is below the preset threshold (desired effect) the system 18 remains in the first mode. If the AI prediction exceeds the predefined effect the system 18 switches into the second mode in order to prevent additional thermal damages. The second mode is characterized in that the output power is sufficiently low in order to avoid causing additional tissue damages (here: 10 W), however high enough in order to allow a valid AI prediction of the attained electrical data within this mode. The output power is thereby so high that in the second mode a stable plasma can be maintained in order to pick electrical data that serve as basis for the decision whether it has to be changed into the first mode.

In order to further reduce the energy introduction and the tissue damage resulting therefrom in the second mode the voltage can be pulsed. Pulsing in this case means that the voltage (and the current) switch back and forth between an on-period and an off-period in the second mode. In the on-period a defined low power is applied. During the off-period no power is output to the tissue. In doing so, the total energy introduction is considerably reduced during the second mode, which further reduces the devitalization of the tissue in this condition. The sum of the on-period the off-period can be set to 10 ms; The on-period be set from 2.4 to 10 ms. The remaining time is the off-period in which no current is output. If 10 ms are selected for the on-period, the off-period is 0 ms and no power reduction is carried out so that the low power condition is always in the on-period. The control between the high and low power condition (first and second mode) shall avoid an overdosage (of the electrical current) and shall attain a reproducible and thus homogeneous tissue effect. As soon as the preset tissue effect is achieved, the control module 19 switches into a condition in which less power is available. With this low power no significant tissue effect is created.

The pure latency from the point in time of the measured electrical data over the prediction of the control module 19 until a possible switching of the conditions is approximately 15 ms. Additional latencies in the millisecond range are added, for example due to the transient response of the RF source 18. The control between high and low power condition shall avoid an overdosage (of the electrical current) and shall achieve a reproducible and thus homogeneous tissue effect. As soon as the preset tissue effect is achieved, control module 19 switches into a condition in which less power is available. With this low power no significant tissue effect is produced.

For training the AI electrical data are characterized by the degree of devitalization of the tissue surface. For this purpose images of the treated areas are recorded and grouped (labeled) based on the coloration of the surface. The respective electrical data (that means the pattern formed by the latter) are used in order to train the different groups or labels (that can be selected as effect levels on the user interface subsequently). The color of the treated tissue is selected, because this is the information available for the physician during the surgical application. Due to the used apparatuses (system consisting of an endoscope, video processor, monitor, etc.) the color of the tissue at a specific treatment duration may change from system to system and is not comparable. During recording of the images, however, defined settings are used in order to guarantee that the color of the images is not falsified by the used equipment (for example camera) and that the results are comparable.

For the use of control module 19 no images are required, but only electrical data are used for prediction of the tissue devitalization.

The AI-controlled argon plasma coagulation is only an example for the implementation of an AI-controlled function in a module in which a previously trained (unchangeable) AI algorithm controls the power output. The training of control module 19 based on labels for which visual data of real tissue are compared with the respective electrical data, can be used for different modes/indications, for example shrinking (visual) for the quality of the thermofusion, degree of devitalization (visual coagulation zone, collateral damages) during electrosurgical cutting of tissue, penetration depth (for example during ESD), enlargement of RF ablation zones (visual ablation zone) and electrical data during electro-surgical cutting.

Moreover this method is not limited to electrical data, but can also be extended on additional data, for example for the control of cushion formation in hydro-technology, temperature regulation during RFA or ice ball formation in the cryo technology.

After switching into the second mode with low power the electrical variables of sensors S1, S2, S3 can have different values and can again change over time as illustrated in FIG. 4 by means of dotted curve branches. For example, the electrical current of sensor S1 can first decrease due to the reduction of the power, however can increase slightly again due to re-wetting of the tissue in the temporal progress. Likewise the variables monitored by the other sensors S2, S3 can change again over time. Also the pattern created in this manner can be comprised in the training data sets and thus serve for the control module 19 to verify the attained effect according to the desired effect label.

With the generator 10 according to the invention the treatment of biological tissue by means of electrosurgical instruments, particularly by means of argon plasma probes, can be carried out reliably without depending on the personal skills of a treating person. With the aid of image-supported measurement value generation, for example using a camera or a medical imaging device, such as CT, a multiplicity of test treatments of tissue samples is carried out and on this basis a training data set is created. From the training data set based on machine learning a control data set is created that controls during subsequent use an apparatus 10 located in an operation room without the aid of a camera observation of the field of operation. Only the typical patterns of sensor data are evaluated that have been assigned to specific tissue effects during the camera-monitored training sessions.

1. A generator, comprising:
a control module having inputs to which only sensors for electrical variables are connected;
an electrical source connected to the control module and adapted to be controlled by the control module and that is connected with a medical instrument and adapted to supply the medical instrument with electrical power,
wherein the control module comprises a control data set, which is based on a training data set, that comprises image data and electrical variables that are detected by means of the sensors.

2. The generator according to claim 1, wherein the control module is adapted to operate the electrical source either in a first mode (HIGH) or in a second mode (LOW), wherein the electrical source provides a high power in the first mode (HIGH) and a low power in the second mode (LOW), the high power being greater than the low power.

3. The generator according to claim 2 wherein in the electrical power of the generator in the first mode (HIGH) is dimensioned for attaining a devitalization and coagulation of biological tissue and that the electrical power of the generator in the second mode (LOW) is dimensioned to avoid devitalization and coagulation of the biological tissue however sufficiently high in order to provide a stable plasma ignition and a valid data determination.

4. The generator according to claim 1, wherein the control data set is created by machine learning on the basis of the training data set with image data.

5. The generator according to claim 1 wherein, the image data are individual images, image sequences, and/or video data and that the electrical variables include multiple measurement points determined in time intervals as individual measurement values or are temporal progresses of the electrical variables.

6. The generator according to claim 4, wherein the control data set comprises effect labels obtained from a manual evaluation of tissue test treatment results.

7. The generator according to claim 5, wherein the control data set comprises effect labels that characterize different degrees of tissue devitalization and/or different penetration depths of the tissue effect.

8. The generator according to claim 2, wherein the control module is configured to switch from the first mode (HIGH) to the second mode (LOW) upon recognition of a pattern of the electrical variables that is assigned to a desired effect label.

9. The generator according to claim 8, wherein the control module is configured to continue monitoring the pattern of the electrical variables after switching to the second mode (LOW).

10. The generator according to claim 9, wherein the control module is configured to switch from the second mode (LOW) to the first mode (HIGH) if the pattern recorded in the second mode (LOW) corresponds to an effect label that is too low.

11. The generator according to claim 9, wherein the control module is configured to switch from the second mode (LOW) into a third mode (OFF) if the pattern recorded in the second mode (LOW) corresponds to an effect label that is too high.

12. The generator according to claim 1, wherein the control module comprises a distance measurement function.

13. The generator according to claim 1, wherein the control module is configured to determine a distance between the instrument and a biological object based on electrical variables detected by the sensors.

14. The generator according to claim 13, wherein the control module is configured to determine the distance based on a non-linearity of a load that is effective at the output of the generator.

15. A method for generation of a control data set of a control module of an electrosurgical generator and subsequent operation of such a generator, the method comprising:
during a training process, generating a training data set by influencing a biological tissue using the generator in a predefined setting and thereby provided or resulting electrical variables are determined as well as resulting tissue changes are recorded by means of an imaging device,
assigning effect labels the tissue changes,
determining a control data set from the training data set using machine learning, wherein the control data set represents a relation between the electrical variables and the effect labels,
controlling the generator based on the control data set during an application procedure for attaining a desired effect corresponding to a selected effect label.

1. A method treatment of the mucosa using an electrosurgical generator that operates an electrosurgical instrument, the method comprising:
providing a control data set associating at least one preset treatment effect to a plurality of electrical variables of the electrosurgical generator;
selecting one of the at least one preset treatment effect;
initiating the treatment of the mucosa by the electrosurgical instrument;
detecting the plurality of electrical variables during the treatment and determining a current tissue effect based on the plurality of electrical variables and the control data set;
reducing a power output of the electrosurgical generator when the current tissue effect meets the selected one of the at least one preset treatment effects.

16. The method of claim 16, wherein:
the at least one preset treatment effect includes a treatment depth, selecting one of the at least one preset treatment effect comprises selecting a desired penetration depth, the current tissue effect includes a current penetration depth, and the “reducing a power output” step comprises reducing a power output of the electrosurgical generator when the current penetration depths meets the selected desired penetration depth.

17. The method of claim 17, further comprising:
moving the electrosurgical instrument to another area of the mucosa;
re-determining the current tissue effect based on the plurality of electrical variables and the control data set;
increasing the power output of the electrosurgical generator when the current penetration depth has not met the desired penetration depth.

18. The method of claim 16, where the at least one preset treatment effect includes a degree of devitalization.

19. The method of claim 16, wherein the at least one preset treatment effect includes ablation and the treatment includes the application of plasma to the mucosa in a gastrointestinal tract for the treatment of obesity.

20. The method of claim 17, wherein the desired penetration depth extends beyond the mucosa up to about two-thirds of a submucosa adjacent the mucosa.

21. The method of claim 17, wherein the desired penetration depth is set manually.

22. The method of claim 16, further comprising: generating the control data set by:
influencing a biological tissue using the electrosurgical generator in a predefined setting;
recording the plurality of electrical variables during the influencing step and associating the recorded plurality of electrical variables with resulting tissue changes, the resulting tissue changes recorded via an imaging device.

23. The method of claim 17, further comprising automatically switching the electrosurgical generator from a first mode to a second mode when the detected plurality of electrical variables correspond, based on the control data set, to the selected one of the at least one preset treatment effect, the second mode providing a lower power than the first mode and being insufficient to further increase the current penetration depth.

24. The method of claim 24, further comprising: moving the electrosurgical instrument over the mucosa to treat a large area while the automatic switching between the first and second modes controls the treatment depth across the area.
